(19) United States
(12) Patent Application Publication
(10) Pub. No.: US 2023/0114848 A1
(43) Pub. Date: Apr. 13, 2023

(54) BONE INFORMATION ACQUISITION APPARATUS, NOTIFICATION APPARATUS, X-RAY DIAGNOSIS APPARATUS, BONE INFORMATION ACQUISITION METHOD, AND STORAGE MEDIUM

(71) Applicant: CANON KABUSHIKI KAISHA, Tokyo (JP)

(72) Inventors: Haruki Iwai, Tochigi (JP); Sota Torii, Tokyo (JP); Daisuke Sato, Tochigi (JP)

(21) Appl. No.: 17/938,630

(22) Filed: Oct. 6, 2022

(30) Foreign Application Priority Data
Oct. 7, 2021 (JP) 2021-165334

(51) Int. Cl. A61B 6/00; G06T 7/73; G06T 7/00; G06T 5/50; G06T 5/00

(52) U.S. Cl. CPC A61B 6/505; A61B 6/4291; G06T 5/002; G06T 5/50; G06T 7/0012; G06T 7/73; G06T 2207/10116; G06T 2207/30008

(57) ABSTRACT

A bone information acquisition apparatus comprises: an X-ray image acquisition unit configured to acquire an X-ray image with a subject; an identifying unit configured to identify a mounted state of a grid that removes scattered rays; and a bone information acquisition unit configured to acquire bone information that evaluates a state of a bone of the subject included in the X-ray image in accordance with the mounted state of the grid.

### BACKGROUND OF THE DISCLOSURE

### Field of the Disclosure

The embodiments disclosed in this specification and the accompanying drawings relate to a bone information acquisition apparatus, a notification apparatus, an x-ray diagnosis apparatus, a bone information acquisition method, and a computer-readable storage medium.

### Description of the Related Art

There is known a technique of acquiring bone information representing the state of the bone of a subject, such as a bone mineral density (BMD) or bone mineral content (BMC), based on an X-ray image of the subject. A grid is sometimes mounted on an X-ray detector to remove the influence of scattered rays (secondary X-rays) reaching the image receiving unit of the X-ray detector.

The embodiments disclosed in this specification and the accompanying drawings improve the acquisition accuracy of bone information representing the state of the bone of a subject.

### SUMMARY OF THE DISCLOSURE

According to one aspect of the present disclosure, there is provided a bone information acquisition apparatus comprising: an X-ray image acquisition unit configured to acquire an X-ray image with a subject; an identifying unit configured to identify a mounted state of a grid that removes scattered rays; and a bone information acquisition unit configured to acquire bone information that evaluates a state of a bone of the subject included in the X-ray image in accordance with the mounted state of the grid.

Further features of the present disclosure will become apparent from the following description of exemplary embodiments (with reference to the attached drawings).

### BRIEF DESCRIPTION OF THE DRAWINGS

FIG. 1 is a block diagram showing an example of the arrangement of an X-ray diagnosis apparatus according to the first embodiment;

FIG. 2 is a flowchart showing a series of processing by a bone information acquisition apparatus according to the first embodiment;

FIG. 3 is a view showing an example of grid stripes according to the first embodiment;

FIG. 4 is a flowchart for explaining determination processing according to the first embodiment;

FIG. 5 is a block diagram showing an example of the arrangement of an X-ray diagnosis apparatus according to the second embodiment;

FIG. 6 is a flowchart showing a series of processing by a bone information acquisition apparatus according to the second embodiment;

FIG. 7 is a flowchart for explaining determination processing according to the second embodiment;

FIG. 8 is a view for explaining notifications according to the second embodiment;

FIG. 9 is a block diagram showing an example of the arrangement of an X-ray diagnosis apparatus according to the third embodiment;

FIG. 10 is a flowchart showing a series of processing by a bone information acquisition apparatus according to the third embodiment;

FIG. 11 is a flowchart for explaining determination processing according to the third embodiment;

FIG. 12 is a block diagram showing an example of a medical information processing system according to the fourth embodiment; and

FIG. 13 is a block diagram showing an example of the arrangement of the medical information processing system according to the fourth embodiment.

### DESCRIPTION OF THE EMBODIMENTS

Hereinafter, embodiments will be described in detail with reference to the attached drawings. Note, the following embodiments are not intended to limit the scope of the claimed invention. Multiple features are described in the embodiments, but limitation is not made to any specific embodiment that requires all such features, and multiple such features may be combined as appropriate. Furthermore, in the attached drawings, the same reference numerals are given to the same or similar configurations, and redundant description thereof is omitted.

### First Embodiment

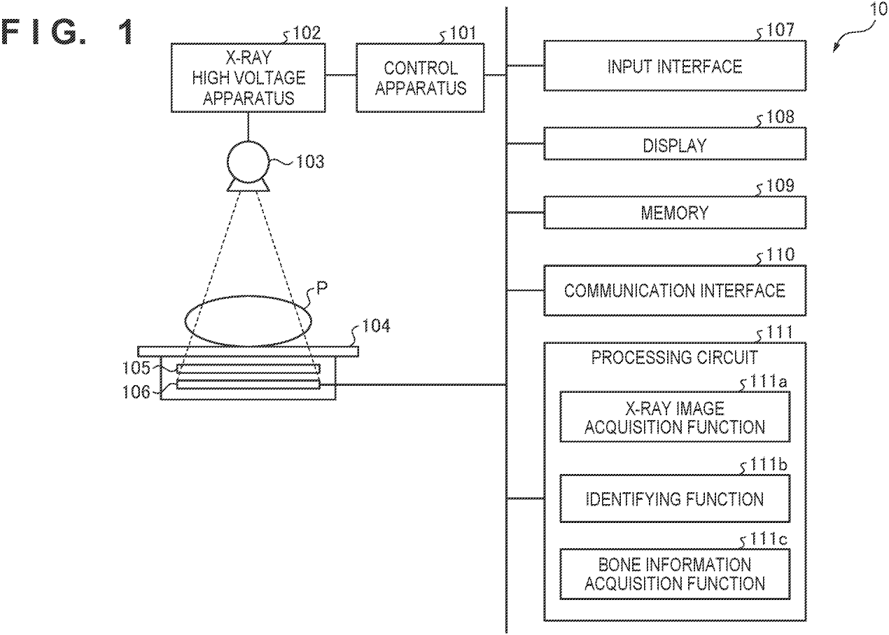

The arrangement of an X-ray diagnosis apparatus 10 will be described with reference to FIG. 1. The X-ray diagnosis apparatus 10 includes a control apparatus 101, an X-ray high voltage apparatus 102, an X-ray tube 103, a top 104, a grid 105, an X-ray detector 106, an input interface 107, a display 108, a memory 109, a communication interface 110, and a processing circuit 111.

The control apparatus 101 drives the X-ray high voltage apparatus 102. In addition, the control apparatus 101 receives an X-ray detection result (detection signal) from the X-ray detector 106 and supplies the detection result to the processing circuit 111.

The X-ray high voltage apparatus 102 applies a high voltage to the X-ray tube 103 under the control of the control apparatus 101. The X-ray tube 103 is a vacuum tube including a cathode having a filament and an anode having a target. The X-ray tube 103 emits thermions from the filament to the target using the high voltage applied from the X-ray high voltage apparatus 102 and causes the thermions to impinge on the target, thereby generating X-rays.

The X-ray tube 103 includes aperture blades formed from an X-ray shielding material such as lead or tungsten. The aperture blades serve to narrow the X-rays generated by the target and are slidably provided. The X-rays generated by the target pass through an opening formed by, for example, four aperture blades and irradiates a subject P.

The top 104 is a bed on which the subject P is placed. A gantry having the top 104 has a drive mechanism such as a motor or actuator. The top 104 is provided so as to be movable and tiltable in the horizontal direction and the height direction by the drive mechanism.

The grid 105 removes scattered rays (secondary X-rays) generated by irradiation of the subject P with X-rays and is provided so as to be detachable at a position between the top 104 and the X-ray detector 106. The grid 105 is formed from an X-ray shielding material such as lead or tungsten and formed in a lattice pattern.

The grid 105 varies in type, including a single grid, a cross grid, and a honeycomb grid. The single grid is a grid with grid elements formed in one direction. The cross grid is a grid with grid elements formed so as to intersect with each other. The honeycomb grid is a grid with grid elements formed in a honeycomb pattern (hexagonal pattern). The grids 105 of different types have different effects of removing scattered rays.

The X-ray detector 106 is formed from, for example, a flat panel detector (FPD). The X-ray detector 106 detects the X-rays emitted from the X-ray tube 103 and transmitted through the subject P and the grid 105. The X-ray detector 106 supplies a detection signal corresponding to the detected X-ray dose to the control apparatus 101. Note that the X-ray detector 106 may have a structure formed by stacking two types of phosphors having different X-ray absorption sensitivities, such as cesium iodide (CsI) and gadolinium oxide sulfide (GOS). This makes it possible to collect X-ray images (dual energy) with two types of energies by one irradiation with X-rays. The X-ray detector 106 may be either of an indirect conversion type or a direct conversion type.

The input interface 107 is implemented by, for example, a mouse and a keyboard, a trackball, switches, buttons, a joystick, a touch pad that performs input operations based on touches on the operation screen, a touch screen as a combination of a display screen and a touch pad, a non-contact input circuit using an optical sensor, a speech input circuit, and the like. The input interface 107 receives various types of input operations from the user and supplies electrical signals corresponding to the received input operations to the processing circuit 111.

The display 108 is formed from, for example, a display apparatus such as a liquid crystal display or a cathode ray tube (CRT) display. The display 108 displays various types of information supplied from the processing circuit 111.

The memory 109 is formed from, for example, a random access memory (RAM), a semiconductor memory device such as a flash memory, a hard disk, an optical disk, or the like. The memory 109 stores various types of information supplied from the processing circuit 111. The memory 109 also stores programs to be executed by the processing circuit 111.

The communication interface 110 is formed from, for example, a network card or network adapter. The communication interface 110 transmits and receives various types of information to and from external apparatuses connected via a network under the control of the processing circuit 111.

The processing circuit 111 is formed from an arithmetic processing apparatus such as a central processing unit (CPU) or micro-processing unit (MPU). The processing circuit 111 controls the overall X-ray diagnosis apparatus 10 by controlling each unit of the X-ray diagnosis apparatus 10.

The processing circuit 111 reads out programs stored in the memory 109 and executes them to function as an X-ray image acquisition function 111a, an identifying function 111b, and a bone information acquisition function 111c. The X-ray image acquisition function 111a is an example of an X-ray image acquisition unit. The identifying function 111b is an example of an identifying unit. The bone information acquisition function 111c is an example of a bone information acquisition unit.

The X-ray diagnosis apparatus 10 having the above arrangement performs the processing of acquiring bone information representing the state of the bone of the subject P, such as a bone mineral density (BMD) and a bone mineral content (BMC), based on the X-ray images obtained by performing X-ray imaging with respect to the subject P.

The X-rays detected by the X-ray detector 106 vary in type in accordance with the mounted state of the grid 105. That is, the effect of removing scattered rays included in X-rays reaching the X-ray detector 106 changes in accordance with the presence/absence of the grid 105 and the type of the grid 105. For this reason, in order to accurately acquire bone information representing the state of the bone of the subject P, such as a bone mineral density and bone mineral content, based on X-ray images, it is necessary to perform proper processing in accordance with the mounted state of the grid 105.

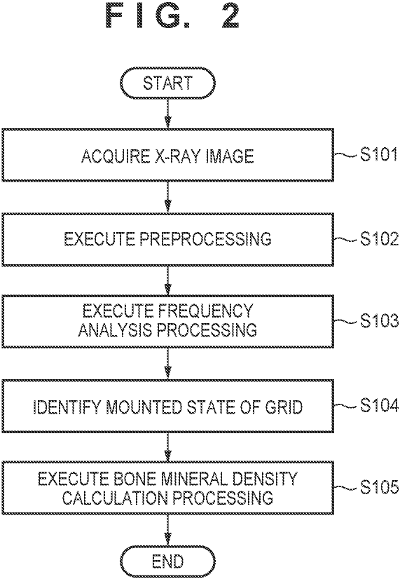

The processing executed by the X-ray diagnosis apparatus 10 will be described below with reference to FIG. 2. The X-ray image acquisition function 111a acquires an X-ray image with the subject P by performing X-ray imaging with respect to the subject P (step S101). The X-ray image acquisition function 111a acquires X-ray images with the subject P which correspond to a plurality of energies (tube voltages). More specifically, the X-ray image acquisition function 111a acquires an X-ray image with the subject P which corresponds high energy (for example, 140 kV) and an X-ray image with the subject P which corresponds to low energy (for example, 80 kV).

The X-ray image acquisition function 111a acquires an X-ray image (gain image) for calibration without the subject P by performing X-ray imaging without the grid 105. For example, the memory 109 stores in advance a high-energy gain image and a low-energy gain image, and the X-ray image acquisition function 111a reads out each energy gain image from the memory 109.

The identifying function 111b executes preprocessing for the X-ray images acquired in step S101 (step S102). The preprocessing includes gain correction, offset correction, and defect correction. For example, the identifying function 111b performs gain correction for an X-ray image corresponding to high energy (140 kV) based on the high-energy gain image. The identifying function 111b also performs gain correction for an X-ray image corresponding to low energy (80 kV) based on the low-energy gain image. With this processing, for example, the identifying function 111b corrects variations in X-ray sensitivity for each detection element of the X-ray detector 106.

The identifying function 111b executes frequency analysis processing (step S103) for an X-ray image after preprocessing and determines whether frequency components unique to the grid 105 have occurred in the horizontal and vertical directions. Note that frequency analysis processing may be performed for at least the X-ray image corresponding to high energy or the X-ray image corresponding to low energy. When the grid 105 is mounted at the time of X-ray imaging, grid stripes corresponding to the type of the grid 105 appear in an X-ray image, and the X-ray image includes frequency components unique to the grid 105. For example, if the mounted grid 105 is a single grid, grid stripes appear in an X-ray image in one direction. If the mounted grid 105 is a cross grid, intersecting grid stripes appear in an X-ray image, for example, as shown in FIG. 3.

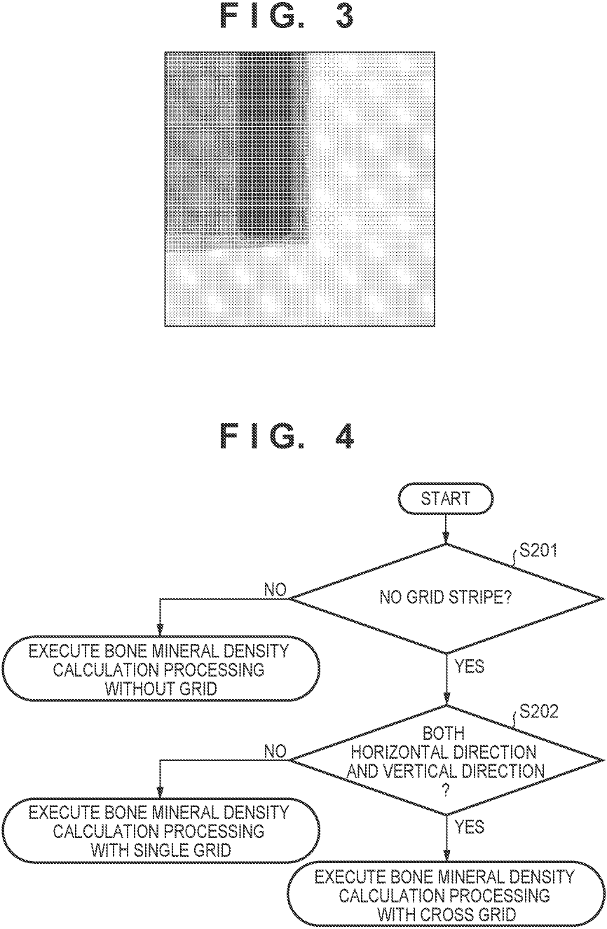

The identifying function 111b identifies the mounted state of the grid 105 from the result of the frequency analysis processing in step S103 (step S104). For example, as shown in FIG. 4, the identifying function 111b identifies the presence/absence of the mounted grid 105 in accordance with whether grid stripes are included in an X-ray image with the subject P (an X-ray image after preprocessing) (step S201).

If grid stripes are included (YES in step S201), the identifying function 111b identifies that the grid 105 is mounted. In this case, the identifying function 111b identifies the type of the grid 105 based on the direction of the grid stripes. The identifying function 111b identifies whether the type of the grid 105 is a single grid or cross grid, in accordance with whether the grid stripes have one direction or intersecting directions (step S202). If grid stripes appear in both the horizontal direction and the vertical direction, the identifying function 111b identifies the type of the grid 105 as a cross grid (YES in step S202). If grid stripes do not appear in both the horizontal direction and the vertical direction (grid stripes appear in only one direction), the identifying function 111b identifies the type of the grid 105 as a single grid (NO in step S202). If no grid stripe is included (NO in step S201), the identifying function 111b identifies that the grid 105 is not mounted.

The identifying function 111b identifies the density of the grid stripes included in an X-ray image with the subject P (an X-ray image after preprocessing). The identifying function 111b also identifies the position of the grid stripes included in an X-ray image.

The bone information acquisition function 111c executes bone mineral density calculation processing in accordance with the result obtained in step S104 (step S105). If, for example, it is identified in step S104 that the grid 105 is not mounted, the bone information acquisition function 111c executes bone mineral density calculation processing without the grid. If it is identified in step S104 that the single grid is mounted, the bone information acquisition function 111c executes bone mineral density calculation processing with the single grid. If it is identified in step S104 that the cross grid is mounted, the bone information acquisition function 111c executes bone mineral density calculation processing with the cross grid.

The bone information acquisition function 111c performs image correction to remove scattered rays from an X-ray image with the subject P (an X-ray image after preprocessing) in bone mineral density calculation processing without the grid. The bone information acquisition function 111c performs image correction to remove grid stripes and scattered rays from the X-ray image with the subject P (the X-ray image after preprocessing) in bone mineral density calculation processing with the single grid. The bone information acquisition function 111c performs image correction to remove grid stripes and scattered rays from the X-ray image with the subject P (the X-ray image after preprocessing) in bone mineral density calculation processing with the cross grid.

The bone information acquisition function 111c acquires bone information for evaluating the state of the bone of the subject P included in an X-ray image in accordance with the mounted state of the grid 105. For example, the bone information acquisition function 111c changes the way of acquiring bone information in accordance with the presence/absence of the mounted grid 105. The bone information acquisition function 111c changes the intensity of scattered ray correction for an X-ray image with the subject P in accordance with the presence/absence of the mounted grid 105. That is, since the amount of scattered rays included in an X-ray image differs in accordance with the presence/absence of the mounted grid 105, the bone information acquisition function 111c may increase the intensity of scattered ray correction when the grid 105 is not mounted as compared with when the grid 105 is mounted.

The bone information acquisition function 111c changes the way of acquiring bone information in accordance with the type of the grid 105. The bone information acquisition function 111c changes the intensity of scattered ray correction for an X-ray image with the subject P in accordance with whether the type of the grid 105 is a single grid or double grid. That is, since the amount of scattered rays included in an X-ray image differs in accordance with whether the type of the grid 105 is a single grid or cross grid, the bone information acquisition function 111c may increase the intensity of scattered ray correction when the single grid is mounted as compared with when the cross grid is mounted.

The bone information acquisition function 111c changes the intensity of scattered ray correction for an X-ray image with the subject P in accordance with the density of grid stripes identified by the identifying function 111b. That is, since the amount of scattered rays included in the X-ray image differs in accordance with the density of grid stripes, if the density of the grid is relatively low (small), the bone information acquisition function 111c may increase the intensity of scattered ray correction as compared with when the density of the grid is relatively high (large).

The bone information acquisition function 111c also removes grid stripes included in an X-ray image with the subject P in accordance with the position of the grid stripes identified by the identifying function 111b.

The bone information acquisition function 111c performs the various types of image correction described above for X-ray images after preprocessing (X-ray images respectively corresponding to high energy and low energy). The bone information acquisition function 111c generates a bone image representing the bone components of the subject P (an image with bone components discriminated from other tissues) by material decomposition processing with respect to the X-ray images after image correction (the X-ray images respectively corresponding to high energy and low energy). The bone information acquisition function 111c defines calcium (Ca) and water as reference materials and estimates the abundance ratio of calcium to water and the abundance ratio of water to calcium for each pixel. With this processing, the bone information acquisition function 111c generates a bone image representing a bone mineral density distribution.

The bone information acquisition function 111c sets a region of interest with respect to a femoral portion or lumbar portion included in a bone image and acquires a bone mineral density (BMD) in the region of interest. The bone information acquisition function 111c may also change the error range with respect to bone mineral densities in accordance with the mounted state of a grid. For example, the bone information acquisition function 111c changes the intensity of scattered ray correction in image correction to be performed for an X-ray image in accordance with the presence/absence of the mounted grid 105, the type of the grid 105 (single grid or cross grid), or the density of grid stripes (the mounted state of the grid 105). For this reason, the bone information acquisition function 111c may change the error range with respect to acquired bone mineral densities (BMD) in accordance with the mounted state of the grid 105.

The bone information acquisition function 111c displays the result of the bone mineral density calculation processing described above on the display 108. For example, a bone mineral density in the region of interest is displayed on the display 108, together with the error range (error bar). The bone information acquisition function 111c may also transmit the result of the bone mineral density calculation processing to another apparatus via a network. In this case, the result of the bone mineral density calculation processing is displayed on the other apparatus and provided for a user such as a doctor.

Although a case of acquiring a bone mineral density has been described as an example of bone information acquisition processing, the above technique can be applied to a case of acquiring another type of bone information. For example, the bone information acquisition function 111c may acquire a bone mineral density (BMC) in step S105 in FIG. 2.

As described above, according to the first embodiment, it is possible to improve the acquisition accuracy of bone information representing the state of the bone of the subject P.

Although the above embodiment has exemplified the case in which the identifying function 111b executes preprocessing for the X-ray image acquired in step S101 and identifies the mounted state of the grid 105 based on the X-ray image after the preprocessing, the preprocessing may be omitted. In this case, the identifying function 111b may identify the mounted state of the grid 105 based on the X-ray images acquired in step S101 (at least the X-ray image corresponding to high energy or the X-ray image corresponding to low energy).

In addition, although the above embodiment has exemplified the case in which the identifying function 111b identifies the mounted state of the grid 105 based on an X-ray image with the subject P, the X-ray image may be an X-ray image without the subject P.

For example, in step S101 shown in FIG. 2, the X-ray image acquisition function 111a may acquire an X-ray image without the subject P without changing the mounted state of the grid 105 after the acquisition of an X-ray image with the subject P, and the identifying function 111b may identify the mounted state of the grid 105 based on an X-ray image without the subject (at least an X-ray image corresponding to high energy or an X-ray image corresponding to low energy).

Note that the X-ray image acquisition function 111a may acquire an X-ray image without the subject P before the acquisition of an X-ray image with the subject P in step S101 shown in FIG. 2, and may acquire an X-ray image with the subject P without changing the mounted state of the grid 105 after the acquisition of an X-ray image without the subject P. In this case, the identifying function 111b may identify the mounted state of the grid 105 based on an X-ray image without the subject P (at least an X-ray image corresponding to high energy or an X-ray image corresponding to low energy).

Although the above embodiment has exemplified the case in which the identifying function 111b identifies the mounted state of the grid 105 based on an X-ray image with the subject P, the identifying function 111b may identify the mounted state of the grid 105 based on mounted state identification information for identifying the mounted state of the grid instead of using the X-ray image. The mounted state identification information may have information for identifying at least one of the following: the presence/absence of the mounted grid 105, the type of the grid 105 (a single grid or cross grid), the presence/absence of grid stripes, the direction of the grid stripes, and the position of the grid stripes.

In this case, the embodiment includes a mounted state identification information acquisition unit that acquires mounted state identification information for identifying the mounted state of the grid 105. More specifically, a grid holder to which the grid 105 is detachably attached may include a communication function, and the communication function may communicate with the grid 105 to acquire, from the grid 105, information for identifying at least one of the following: the type of the grid 105 (a single grid or cross grid), the presence/absence of grid stripes, the direction of the grid stripes, and the position of the grid stripes. The mounted state identification information acquisition unit is not limited to any specific means as long as it can acquire mounted state identification information, such as a manual input operation by the user or electrical detection on the hardware side.

### Second Embodiment

The second embodiment will exemplify a case in which a notification is made to prompt to mount a grid 105 suitable for the acquisition of bone information for evaluating the state of the bone of a subject P in accordance with the mounted state of the grid 105.

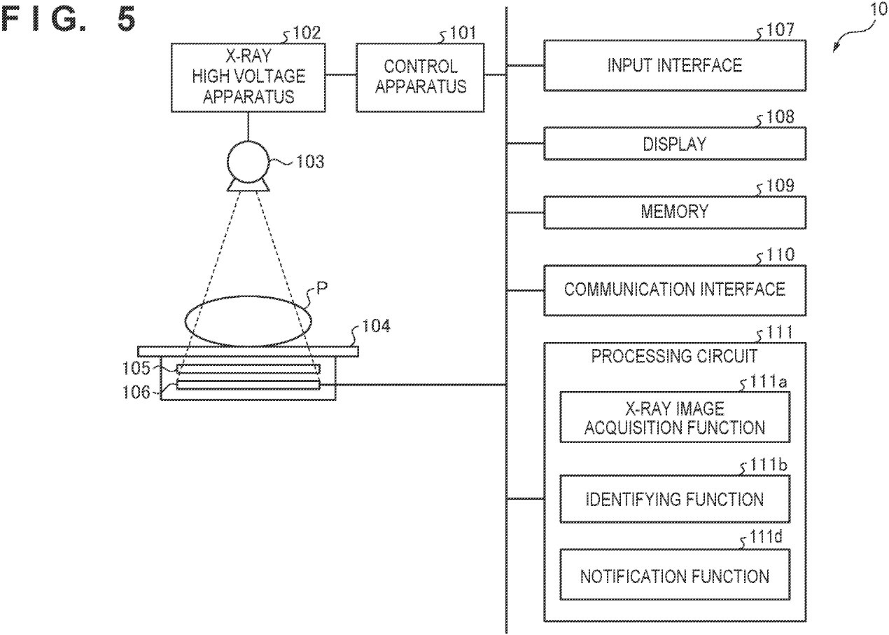

As shown in FIG. 5, a processing circuit 111 according to the second embodiment includes a notification function 111d instead of the bone information acquisition function 111c. The notification function 111d is an example of a notification unit. The same components as those described in the first embodiment will be denoted by the same reference numerals and a description of them will be omitted.

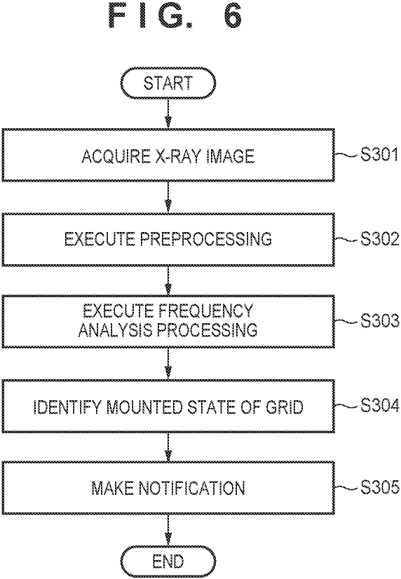

The processing executed by an X-ray diagnosis apparatus 10 will be described below with reference to FIG. 6. The X-ray image acquisition function 111a acquires an X-ray image with the subject P or an X-ray image without the subject P (step S301). An X-ray image may be at least an X-ray image corresponding to high energy or an X-ray image corresponding to low energy.

An identifying function 111b executes preprocessing for the X-ray image acquired in step S101 (at least the X-ray image corresponding to high energy or the X-ray image corresponding to low energy) as in the first embodiment (step S302). Note that the preprocessing may be omitted.

The identifying function 111b executes frequency analysis processing for the X-ray image after the preprocessing as in the first embodiment (step S303) and identifies the mounted state of the grid 105 based on the result of the frequency analysis processing (step S304). For example, as shown in FIG. 4, the identifying function 111b identifies the presence/absence of the mounted grid 105 in accordance with whether the X-ray image includes grid stripes (step S401).

If the X-ray image includes grid stripes (YES in step S401), the identifying function 111b identifies that the grid 105 is mounted. In this case, the identifying function 111b identifies the type of the grid 105 based on the direction of the grid stripes. The identifying function 111b identifies the type of the grid 105 as a single grid or cross grid in accordance with whether the direction of the grid stripes is one direction or intersecting directions (step S402). If the grid stripes appear in both the horizontal direction and the vertical direction, the identifying function 111b identifies that the type of the grid 105 is a cross grid (YES in step S402). If the grid stripes do not appear in both the horizontal direction and the vertical direction (appear in only one of the directions), the identifying function 111b identifies that the type of the grid 105 is a single grid (NO in step S402). If the X-ray image includes no grid stripes in step S201 (NO in step S401), the identifying function 111b identifies that the grid 105 is not mounted.

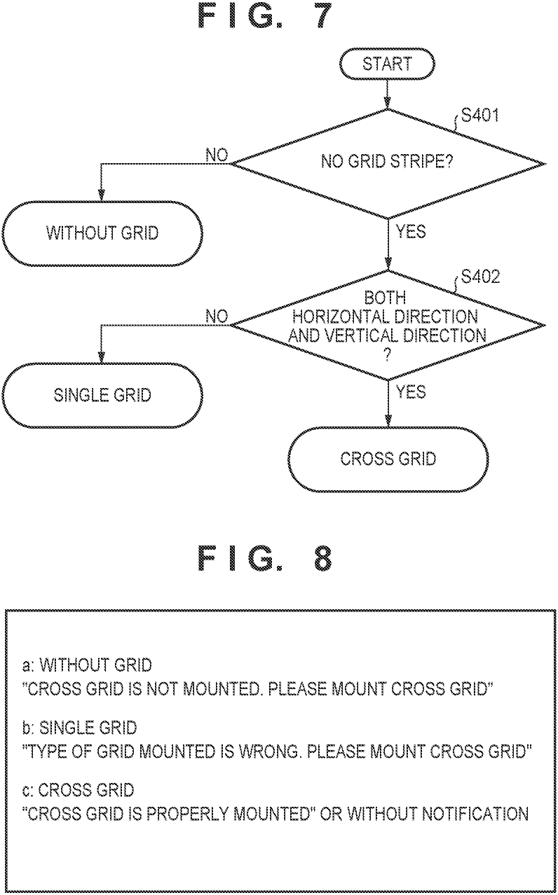

The notification function 111d makes a notification in accordance with the determination result obtained in step S304 (step S305). For example, as shown in FIG. 8, if it is identified in step S401 in FIG. 7 that the grid 105 is not mounted, the notification function 111d causes the display 108 to display the message “the cross grid is not mounted. Please mount the cross grid”. If it is identified in step S402 in FIG. 7 that the type of the grid 105 is a single grid, the notification function 111d causes the display 108 to display the message “the type of grid mounted is wrong. Please mount the cross grid”. If it is identified in step S402 in FIG. 7 that the type of the grid 105 is a single grid, the notification function 111d causes the display 108 to display the message “the cross grid is properly mounted”.

As described above, according to the second embodiment, it is possible to prompt to mount a cross grid having a high scattered ray reducing effect and improve the acquisition accuracy of bone information representing the state of the bone of the subject P.

### Third Embodiment

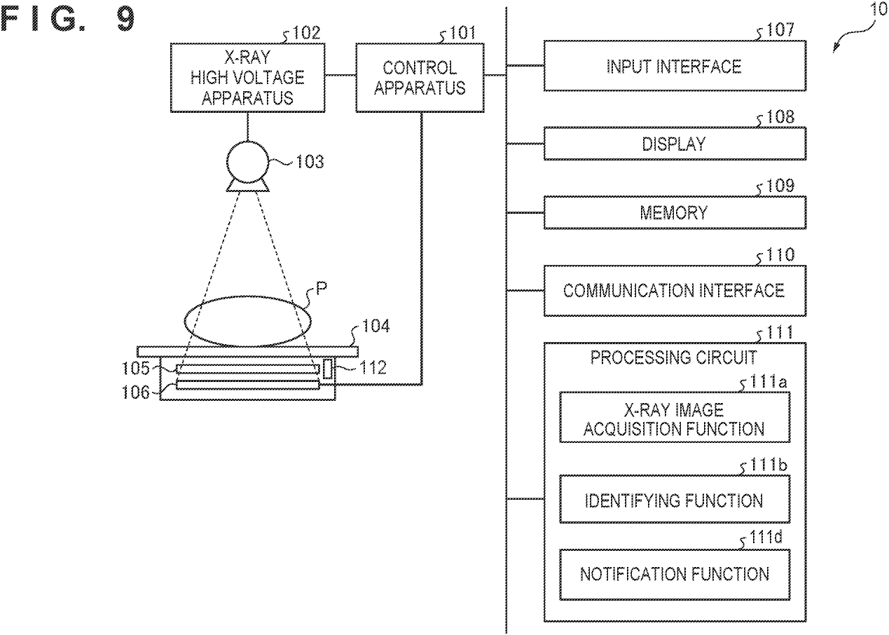

The third embodiment will exemplify a case in which a sensor 112 is provided to detect a grid 105, as shown in FIG. 9. FIG. 9 is a block diagram showing an example of the arrangement of an X-ray diagnosis apparatus 10 according to the third embodiment.

The X-ray diagnosis apparatus 10 according to the third embodiment differs from the X-ray diagnosis apparatuses 10 according to the first and second embodiments in that the sensor 112 is provided, and also differs in part of the processing performed by an identifying function 111b. The same components as those described in the first and second embodiments will be denoted by the same reference numerals and a description of them will be omitted.

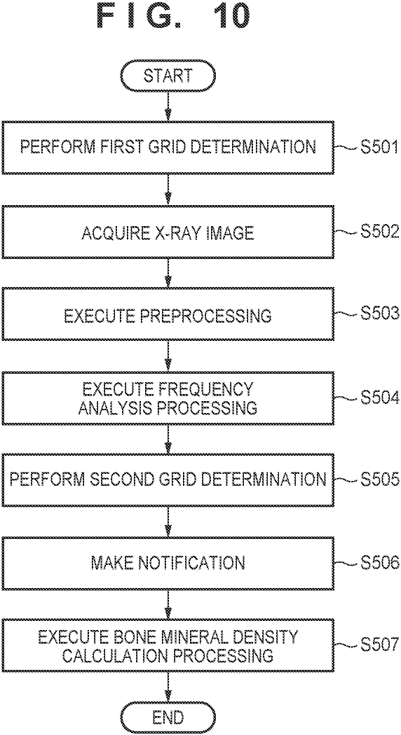

The processing performed by the X-ray diagnosis apparatus 10 according to the third embodiment will be described below with reference to FIG. 10. FIG. 10 is a flowchart showing a series of processing by the X-ray diagnosis apparatus 10 according to the third embodiment.

First of all, the X-ray diagnosis apparatus 10 performs the first grid determination using the sensor 112 (step S501). More specifically, the identifying function 111b determines, from the detection result on the grid 105 by the sensor 112, whether the grid 105 was mounted at the time of the collection of X-ray images. Although the type of the sensor 112 is not specifically limited, for example, examples of the sensor 112 can be a contact sensor, a magnetic sensor, and a pressure sensor. If it is determined in step S501 that the grid 105 is not mounted, the processing from step S502 to step S505 may be omitted.

The X-ray diagnosis apparatus 10 acquires X-ray images (step S502). For example, an X-ray image acquisition function 111a acquires a first X-ray image of a subject P which corresponds to high energy and a second X-ray image of the subject P which corresponds to low energy. The X-ray image acquisition function 111a also acquires a third X-ray image corresponding to high energy and a fourth X-ray image corresponding to low energy collected without any subject.

The X-ray diagnosis apparatus 10 executes preprocessing for the X-ray images acquired in step S502 (step S503). For example, the identifying function 111b executes preprocessing for either the third X-ray image or the fourth X-ray image collected without any subject. Examples of the preprocessing in step S503 can be offset correction, gain correction, or defect correction.

The X-ray diagnosis apparatus 10 then executes frequency analysis processing for the X-ray image after the preprocessing (step S504). For example, the identifying function 111b executes frequency analysis processing for the third X-ray image or the fourth X-ray image after the preprocessing. In this case, the identifying function 111b may execute frequency analysis processing only in a direction parallel to grid stripes that appear when a single grid is mounted as the grid 105. That is, the identifying function 111b may execute frequency analysis processing only in one direction.

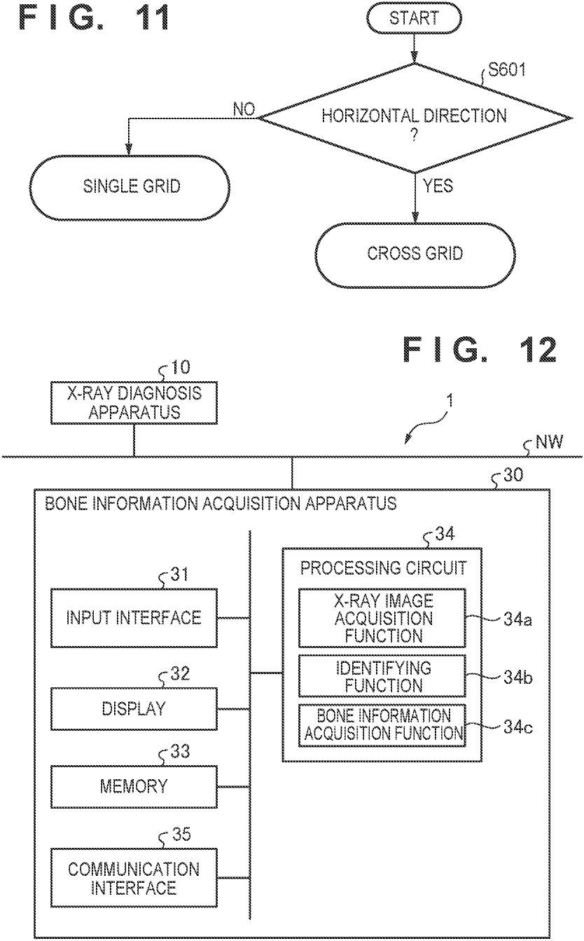

The X-ray diagnosis apparatus 10 then performs the second grid determination based on the result of the frequency analysis processing in step S504 (step S505). The details of the processing in step S505 will be described with reference to FIG. 11. FIG. 11 is a flowchart for explaining determination processing according to the third embodiment.

For example, the identifying function 111b determines, based on the result of the frequency analysis processing, whether grid stripe peaks appear in the horizontal direction (step S601). In this case, if grid stripe peaks appear in the horizontal direction (YES in step S601), the identifying function 111b determines that a cross grid is mounted as the grid 105. In contrast to this, if grid stripe peaks do not appear in the horizontal direction (NO in step S601), the identifying function 111b determines that a single grid is mounted as the grid 105.

The X-ray diagnosis apparatus 10 then makes a notification in accordance with the determination results obtained in step S501 and step S505 (step S506). For example, the notification function 111d can make a notification in step S506 in the same manner as in FIG. 8.

Although not shown in FIG. 9, a processing circuit 111 according to the third embodiment may further include a bone information acquisition function 111c. That is, the processing circuit 111 may further execute the bone mineral density calculation processing described in step S507 in FIG. 10. Alternatively, an apparatus different from the X-ray diagnosis apparatus 10 may execute the bone mineral density calculation processing described in step S507 in FIG. 10.

The bone mineral density calculation processing in step S507 may be executed only if it is determined in step S501 and step S505 that a cross grid is mounted. That is, the bone mineral density calculation processing in step S507 may be executed when a cross grid is mounted as the grid 105, whereas when another type of the grid 105 is mounted or the grid 105 is not mounted, the process may shift again to step S501. In addition, in step S507, bone mineral density calculation processing may be executed in accordance with the determination results obtained in step S501 and step S505. When the bone mineral density calculation processing is executed, the display 108 may display the result of the bone mineral density calculation processing.

As described above, according to the third embodiment, the identifying function 111b determines the mounted state of the grid 105 based on the result of the frequency analysis processing and the detection result on the 105 by the sensor 112. This makes it possible to improve the acquisition accuracy of a bone mineral density and reduce the calculation load for the determination of the mounted state of the grid 105 as compared with the case in which the mounted state of the grid 105 is determined based on only frequency analysis processing. That is, when frequency analysis processing is to be executed only in the horizontal direction in step S504 in FIG. 10, frequency analysis processing in the vertical direction can be omitted. In addition, it is determined in step S505 whether grid stripe peaks appear in the horizontal direction, and determination whether grid stripe peaks appear in the vertical direction can be omitted.

### Fourth Embodiment

According to the above description of the fourth embodiment, the X-ray image acquisition function 111a acquires the third X-ray image corresponding to high energy and the fourth X-ray image corresponding to low energy collected without any subject. However, the embodiment is not limited to this, and the X-ray image acquisition function 111a may acquire only the third X-ray image or the fourth X-ray image.

The above embodiments have exemplified the cases in which steps S102 to S104 in FIG. 2, steps S302 to S304 in FIG. 6, and steps S503 to S505 in FIG. 10 are executed based on the third X-ray image or the fourth X-ray image. However, the embodiments are not limited to this. For example, steps S102 to S104 in FIG. 2, steps S302 to S304 in FIG. 6, and steps S503 to S505 in FIG. 10 may be executed based on the first X-ray image of the subject P which corresponds to high energy or the second X-ray image of the subject P which corresponds to low energy.

The subject P described in each embodiment described above is not limited to a patient and may be a phantom. That is, a bone mineral density is required to be acquired with high accuracy for osteoporosis diagnosis, drug efficacy determination, or the like. Accordingly, in order to guarantee high acquisition accuracy, a quality check using a dedicated phantom embedded with a simulated bone is sometimes executed before the acquisition of a bone mineral density. Applying the X-ray image acquired using the phantom to each embodiment described above can improve the accuracy of bone information representing the state of the simulated bone in a quality check.

When a quality check using a phantom and the acquisition of bone information are successively performed, bone information acquisition processing may be performed by using an X-ray image different from an X-ray image used for the identification of the mounted state of a grid. For example, an X-ray image of a phantom is acquired in step S301 in FIG. 6, and grid determination in steps S302 to S304 is performed based on the X-ray image. In addition, a notification is made in step S305. The acquisition of an X-ray image of the patient and bone information acquisition processing may be performed after a cross grid with high scattered ray reducing effect is mounted based on the notification result.

The above embodiments have exemplified the case in which the first X-ray image and the second X-ray image are acquired as X-ray images corresponding to a plurality of X-ray energies. However, the embodiments are not limited to this, and the X-ray image acquisition function 111a may acquire one X-ray image as long as bone mineral density calculation processing can be executed. For example, when the X-ray detector 106 is a photon counting detector, each embodiment described above can be executed based on one X-ray image.

The above embodiments have exemplified the cases in which preprocessing is executed in step S102 in FIG. 2, step S302 in FIG. 6, step S503 in FIG. 10, and the like. However, the embodiments are not limited to this, and such preprocessing may be omitted as long as grid stripe peaks can be identified by frequency analysis.

The above embodiments have exemplified the case in which the X-ray diagnosis apparatus 10 determines the mounted state of the grid 105. However, the embodiments are not limited to this. That is, an apparatus different from the X-ray diagnosis apparatus 10 may determine the mounted state of the grid 105 in the X-ray diagnosis apparatus 10.

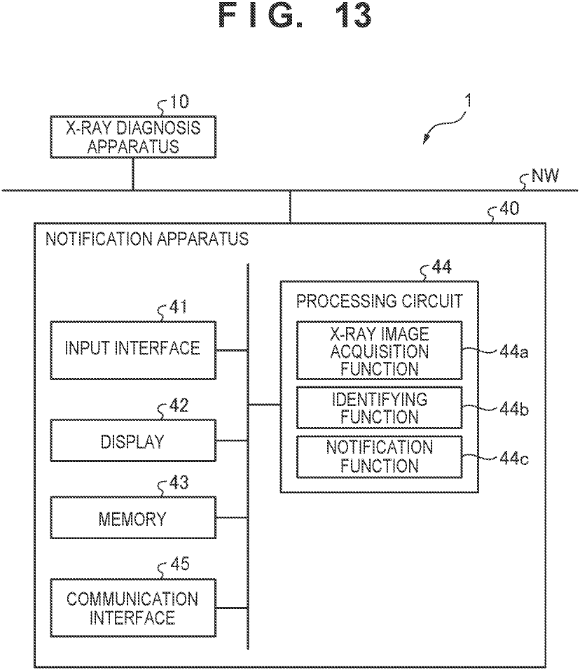

For example, as shown in FIG. 12, the X-ray diagnosis apparatus 10 may be connected to a bone information acquisition apparatus 30 via a network NW, and a processing circuit 34 of the bone information acquisition apparatus 30 may execute an X-ray image acquisition function 34a, an identifying function 34b, and a bone information acquisition function 34c.

The X-ray image acquisition function 34a acquires X-ray images from the X-ray diagnosis apparatus 10 via the network NW. Note that X-ray images may be acquired from the X-ray diagnosis apparatus 10 via another apparatus. For example, the X-ray diagnosis apparatus 10 transmits the acquired X-ray images to an image archiving apparatus such as a picture archiving and communication system (PACS). In this case, the X-ray image acquisition function 34a may acquire X-ray images from the image archiving apparatus.

The identifying function 34b has a function similar to the identifying function 111b and identifies the mounted state of a grid based on an acquired X-ray image. The bone information acquisition function 34c has a function similar to the identifying function 111b and executes the acquisition of bone information for evaluating the state of the bone of the subject P in accordance with the mounted state of the grid 105.

For another example, as shown in FIG. 13, the X-ray diagnosis apparatus 10 is connected to a notification apparatus 40 via the network NW, and a processing circuit 44 of the notification apparatus 40 may execute an X-ray image acquisition function 44a, an identifying function 44b, and a notification function 44c.

The X-ray image acquisition function 44a acquires X-ray images from the X-ray diagnosis apparatus 10 via the network NW. Note that X-ray images may be acquired from the X-ray diagnosis apparatus 10 via another apparatus. For example, the X-ray diagnosis apparatus 10 transmits the acquired X-ray images to an image archiving apparatus such as a PACS. In this case, the X-ray image acquisition function 44a may acquire X-ray images from the image archiving apparatus.

The identifying function 44b is a function similar to the identifying function 111b described above and identifies the mounted state of a grid based on an acquired X-ray image. The notification function 44c is a function similar to the identifying function 111b described above and makes a notification to prompt to mount a grid suitable for the acquisition of bone information for evaluating the state of the bone of the subject P in accordance with the mounted state of the grid 105.

The term “processor” used in the above description refers to, for example, a circuit such as a central processing unit (CPU), a graphics processing unit (GPU), an application specific integrated circuit (ASIC), a programmable logic device (such as a simple programmable logic device (SPLD), a complex programmable logic device (CPLD), a field programmable gate array (FPGA)). When the processor is a CPU, the processor implements a function by reading out and executing a program stored in a storage circuit. When the processor is an ASIC, this function is directly incorporated in the circuit of the processor as a logic circuit instead of the program being stored in the storage circuit. Note that each processor according to the embodiments is not limited to a case in which each processor is configured as a single circuit, and a plurality of independent circuits may be combined into one processor to implement its function. In addition, a plurality of constituent elements in each drawing may be integrated into one processor to implement its function.

In addition, according to the above description, the single memory stores a program corresponding to each processing function of the processing circuit. However, the embodiments are not limited to this. For example, a plurality of memories may be separately arranged, and the processing circuit may read out each program from a corresponding one of the memories. The programs may be directly incorporated in the circuit of the processor instead of being stored in the memory. In this case, the processor implements functions by reading out and executing programs incorporated in the circuit.

The constituent elements of the respective devices according to the above embodiments are functionally conceptual and need not necessarily be configured physically as shown in the accompanying drawings. That is, the specific form of separation/integration of the respective devices is not limited to that shown in the accompanying drawings, and can be functionally or physically separated or integrated partly or wholly according to various types of loads or usages. All or arbitrary some of the respective processing functions executed by the respective devices are implemented by a CPU or programs analytically executed by the CPU or implemented as wired-logic hardware.

According to at least one of the embodiments described above, it is possible to improve the accuracy of bone information representing the state of the bone of a subject.

Concerning the above embodiments, the following supplementary nodes are disclosed as selective features according to one aspect of the present disclosure.

### Supplementary Note 1

There is provided a bone information acquisition apparatus comprising:
an X-ray image acquisition unit configured to acquire an X-ray image with a subject;
an identifying unit configured to identify a mounted state of a grid that removes scattered rays; and
a bone information acquisition unit configured to acquire bone information for evaluating a state of a bone of the subject included in the X-ray image in accordance with the mounted state of the grid.

### Supplementary Note 2

The identifying unit may identify the mounted state of the grid based on the X-ray image with the subject.

### Supplementary Note 3

The X-ray image acquisition unit may acquire an X-ray image without the subject, and

the identifying unit may identify the mounted state of the grid based on the X-ray image without the subject.

### Supplementary Note 4

The identifying unit may identify presence/absence of the mounted grid in accordance with whether the X-ray image includes grid stripes, and

the bone information acquisition unit may change a way of acquiring the bone information in accordance with the presence/absence of the mounted grid.

### Supplementary Note 5

The bone information acquisition unit may change an intensity of scattered ray correction for an X-ray image with the subject in accordance with the presence/absence of the mounted grid.

### Supplementary Note 6

The identifying unit may identify a type of grid based on a direction of grid stripes included in the X-ray image, and

the bone information acquisition unit may change a way of acquiring the bone information in accordance with the type of grid.

### Supplementary Note 7

The identifying unit may identify whether the type of grid is a single grid or cross grid in accordance with whether the direction of the grid stripes is one direction or intersection directions.

### Supplementary Note 8

The bone information acquisition unit may change an intensity of scattered ray correction for an X-ray image with the subject in accordance with whether the type of grid is a single gird or double grid.

### Supplementary Note 9

The identifying unit may identify a density of grid stripes included in the X-ray image, and

the bone information acquisition unit may change an intensity of scattered ray correction for an X-ray image with the subject in accordance with the density of the grid stripes.

### Supplementary Note 10

The identifying unit may identify a position of grid stripes included in the X-ray image, and

the bone information acquisition unit may remove grid stripes included in an X-ray image with the subject in accordance with the position of the grid stripes.

### Supplementary Note 11

The bone information acquisition unit may change an error range with respect to the bone information in accordance with the mounted state of the grid.

### Supplementary Note 12

There is provided a notification apparatus comprising:
an X-ray image acquisition unit configured to acquire an X-ray image with a subject or an X-ray image without the subject;
an identifying unit configured to identify a mounted state of a grid that removes scattered rays based on the acquired X-ray image; and
a notification unit configured to make a notification to prompt to mount a grid suitable for acquisition of bone information that evaluates a state of a bone of the subject in accordance with the mounted state of the grid.

### Supplementary Note 13

The identifying unit identifies presence/absence of the mounted grid in accordance with whether the X-ray image includes grid stripes, and

the notification unit may change a way of making the notification in accordance with presence/absence of the mounted grid.

### Supplementary Note 14

The identifying unit may identify a type of grid based on the direction of grid stripes included in the X-ray image, and

the notification unit may change a way of making the notification in accordance with the type of grid.

### Supplementary Note 15

The identifying unit may identify whether the type of grid is a single grid or cross grid, in accordance with whether the direction of the grid stripes is one direction or intersecting directions.

### Supplementary Note 16

The notification unit may change a way of making the notification in accordance with whether the type of grid is a single grid or double grid.

### Supplementary Note 17

The notification unit may make a notification to prompt to mount a cross grid when the identifying unit identifies that the cross grid is not mounted.

### Supplementary Note 18

There is provided an X-ray diagnosis apparatus comprising:
an X-ray tube configured to irradiate a subject with an X-ray;
an X-ray detector configured to detect X-rays transmitted through the subject;
an X-ray image acquisition unit configured to acquire an X-ray image with the subject;
an identifying unit configured to identify a mounted state of a grid that removes scattered rays; and
a bone information acquisition unit configured to acquire bone information that evaluates a state of a bone of the subject included in the X-ray image in accordance with the mounted state of the grid.

### Supplementary Note 19

There is provided an X-ray diagnosis apparatus comprising:
an X-ray tube configured to irradiate a subject with an X-ray;
an X-ray detector configured to detect X-rays transmitted through the subject;
an X-ray image acquisition unit configured to acquire an X-ray image with a subject or an X-ray image without the subject;
an identifying unit configured to identify a mounted state of a grid that removes scattered rays based on the acquired X-ray image; and
a notification unit configured to make a notification to prompt to mount a grid suitable for acquisition of bone information that evaluates a state of a bone of the subject in accordance with a mounted state of the grid.

### Supplementary Note 20

There is provided a bone information acquisition method comprising causing a processing circuit to
acquire an X-ray image with a subject,
identify a mounted state of a grid that removes scattered rays, and
acquire bone information that evaluates a state of a bone of the subject included in the X-ray image in accordance with the mounted state of the grid.

### Supplementary Note 21

There is provided a notification method comprising causing a processing circuit to
acquire an X-ray image with a subject or an X-ray image without the subject,
identify a mounted state of a grid that removes scattered rays based on the acquired X-ray image, and
make a notification to prompt to mount a grid suitable for acquisition of bone information that evaluates a state of a bone of the subject in accordance with the mounted state of the grid.

### Supplementary Note 22

There is provided a program that causes a computer to execute
acquiring an X-ray image with a subject;
identifying a mounted state of a grid that removes scattered rays; and
acquiring bone information that evaluates a state of a bone of the subject included in the X-ray image in accordance with the mounted state of the grid.

### Supplementary Note 23

There is provided a program that causes a computer to execute
acquiring an X-ray image with a subject or an X-ray image without the subject;
identifying a mounted state of a grid that removes scattered rays based on the acquired X-ray image; and
making a notification to prompt to mount a grid suitable for acquisition of bone information that evaluates a state of a bone of the subject in accordance with the mounted state of the grid.

### Supplementary Note 24

The identifying unit may perform image correction for an X-ray image with the subject based on an X-ray image acquired by X-ray imaging without the subject and without mounting the grid.

### Supplementary Note 25

The identifying unit may determine the presence/absence and type of the grid as the mounted state.

### Supplementary Note 26

The identifying unit may further obtain the density of the grid as the mounted state.

### Supplementary Note 27

The identifying unit may execute preprocessing for the X-ray image and execute the frequency analysis processing for the X-ray image after the preprocessing.

### Supplementary Note 28

The identifying unit may execute frequency analysis processing for an X-ray image without the subject.

### Supplementary Note 29

The bone information acquisition unit may acquire the bone information based on the X-ray image corresponding to high energy and the X-ray image corresponding to low energy.

### Supplementary Note 30

The apparatus may include a mounted state identification information acquisition unit configured to acquire mounted state identification information that identifies the mounted state of the grid, and
the identifying unit may identify the mounted state of the grid based on the mounted state identification information.

### Supplementary Note 31

The mounted state identification information acquired by the mounted state identification information acquisition unit may be information that identifies at least one of presence/absence of the mounted grid, a type of the grid, presence/absence of the grid stripes, a direction of the grid stripes, and a position of the grid stripes.

### OTHER EMBODIMENTS

Embodiment(s) of the present disclosure can also be realized by a computer of a system or apparatus that reads out and executes computer executable instructions (e.g., one or more programs) recorded on a storage medium (which may also be referred to more fully as a ‘non-transitory computer-readable storage medium’) to perform the functions of one or more of the above-described embodiment(s) and/or that includes one or more circuits (e.g., application specific integrated circuit (ASIC)) for performing the functions of one or more of the above-described embodiment(s), and by a method performed by the computer of the system or apparatus by, for example, reading out and executing the computer executable instructions from the storage medium to perform the functions of one or more of the above-described embodiment(s) and/or controlling the one or more circuits to perform the functions of one or more of the above-described embodiment(s). The computer may comprise one or more processors (e.g., central processing unit (CPU), micro processing unit (MPU)) and may include a network of separate computers or separate processors to read out and execute the computer executable instructions. The computer executable instructions may be provided to the computer, for example, from a network or the storage medium. The storage medium may include, for example, one or more of a hard disk, a random-access memory (RAM), a read only memory (ROM), a storage of distributed computing systems, an optical disk (such as a compact disc (CD), digital versatile disc (DVD), or Blu-ray Disc (BD)™), a flash memory device, a memory card, and the like.

While the present disclosure has been described with reference to exemplary embodiments, it is to be understood that the claims are not limited to the disclosed exemplary embodiments. The scope of the following claims is to be accorded the broadest interpretation so as to encompass all such modifications and equivalent structures and functions.

This application claims the benefit of Japanese Patent Application No.2021-165334, filed Oct. 7, 2021, which is hereby incorporated by reference herein in its entirety.

What is claimed is:

1. A bone information acquisition apparatus comprising:
an X-ray image acquisition unit configured to acquire an X-ray image with a subject;
an identifying unit configured to identify a mounted state of a grid that removes scattered rays; and
a bone information acquisition unit configured to acquire bone information that evaluates a state of a bone of the subject included in the X-ray image in accordance with the mounted state of the grid.

2. The apparatus according to claim 1, wherein the identifying unit identifies the mounted state of the grid based on the X-ray image with the subject.

3. The apparatus according to claim 1, wherein the X-ray image acquisition unit acquires an X-ray image without the subject, and
the identifying unit identifies the mounted state of the grid based on the X-ray image without the subject.

4. The apparatus according to claim 2, wherein the identifying unit identifies presence/absence of the mounted grid in accordance with whether the X-ray image includes grid stripes, and
the bone information acquisition unit changes a way of acquiring the bone information in accordance with the presence/absence of the mounted grid.

5. The apparatus according to claim 2, wherein the bone information acquisition unit changes an intensity of scattered ray correction for an X-ray image with the subject in accordance with the presence/absence of the mounted grid.

6. The apparatus according to claim 2, wherein the identifying unit identifies a type of grid based on a direction of grid stripes included in the X-ray image, and
the bone information acquisition unit changes a way of acquiring the bone information in accordance with the type of grid.

7. The apparatus according to claim 6, wherein the identifying unit identifies whether the type of grid is a single grid or cross grid in accordance with whether the direction of the grid stripes is one direction or intersection directions.

8. The apparatus according to claim 7, wherein the bone information acquisition unit changes an intensity of scattered ray correction for an X-ray image with the subject in accordance with whether the type of grid is a single gird or double grid.

9. The apparatus according to claim 2, wherein the identifying unit identifies a density of grid stripes included in the X-ray image, and
the bone information acquisition unit changes an intensity of scattered ray correction for an X-ray image with the subject in accordance with the density of the grid stripes.

10. The apparatus according to claim 2, wherein the identifying unit identifies a position of grid stripes included in the X-ray image, and
the bone information acquisition unit removes grid stripes included in an X-ray image with the subject in accordance with the position of the grid stripes.

11. The apparatus according to claim 2, wherein the bone information acquisition unit changes an error range with respect to the bone information in accordance with the mounted state of the grid.

12. A notification apparatus comprising:
an X-ray image acquisition unit configured to acquire an X-ray image with a subject or an X-ray image without the subject;
an identifying unit configured to identify a mounted state of a grid that removes scattered rays based on the acquired X-ray image; and
a notification unit configured to make a notification to prompt to mount a grid suitable for acquisition of bone information that evaluates a state of a bone of the subject in accordance with the mounted state of the grid.

13. The apparatus according to claim 1, further comprising an X-ray diagnosis apparatus,
wherein the X-ray diagnosis apparatus comprises:
an X-ray tube configured to irradiate a subject with an X-ray;
an X-ray detector configured to detect X-rays transmitted through the subject;
an X-ray image acquisition unit configured to acquire an X-ray image with the subject;
an identifying unit configured to identify a mounted state of a grid that removes scattered rays; and
a bone information acquisition unit configured to acquire bone information that evaluates a state of a bone of the subject included in the X-ray image in accordance with the mounted state of the grid.

14. A bone information acquisition method comprising causing a processing circuit to
acquire an X-ray image with a subject,
identify a mounted state of a grid that removes scattered rays, and
acquire bone information that evaluates a state of a bone of the subject included in the X-ray image in accordance with the mounted state of the grid.

15. A non-transitory computer-readable storage medium storing a program for causing a computer to execute the method according to claim 14.

16. The apparatus according to claim 1, wherein the identifying unit performs image correction for an X-ray image with the subject based on an X-ray image obtained by X-ray imaging without the subject and without mounting the grid and identifies the mounted state of the grid based on the X-ray image after the image correction.

17. The apparatus according to claim 1, wherein the identifying unit determines presence/absence of the grid and a type of grid as the mounted state,
the identifying unit obtains a density of the grid stripe,
the identifying unit executes preprocessing for the X-ray image and executes frequency analysis processing for the X-ray image after the preprocessing, or
the identifying unit executes frequency analysis processing for an X-ray image without the subject.

18. The apparatus according to claim 1, wherein the bone information acquisition unit acquires the bone information based on the X-ray image corresponding to high energy and the X-ray image corresponding to low energy.

19. The apparatus according to claim 1, further comprising a mounted state identification information acquisition unit configured to acquire a mounted state identification information that identifies the mounted state of the grid,
wherein the identifying unit identifies the mounted state of the grid based on the mounted state identification information.

20. The apparatus according to claim 1, wherein the mounted state identification information acquired by the mounted state identification information acquisition unit is information that identifies at least one of presence/absence of the mounted grid, a type of the grid, presence/absence of the grid stripes, a direction of the grid stripes, and a position of the grid stripes.
